(19) United States
(12) Patent Application Publication
(10) Pub. No.: US 2018/0160999 A1
(43) Pub. Date: Jun. 14, 2018

(54) DIAGNOSTIC IMAGING SYSTEM COMPRISING A DEVICE FOR FACILITATING BREAST EXAMINATIONS

(71) Applicant: NOVAURA S.R.L., 20092 CINISELLO BALSAMO (MI) (IT)

(72) Inventor: DANILO GENNARI, MONZA (IT)

(21) Appl. No.: 15/576,577

(22) PCT Filed: Jun. 1, 2016
(86) PCT No.: PCT/IB2016/053217

§ 371 Date: Nov. 22, 2017

(30) Foreign Application Priority Data
Jun. 3, 2015 (IT) 102015000020135

(51) Int. Cl. A61B 8/08; A61B 8/00

(52) U.S. Cl. CPC A61B 8/0825; A61B 5/0062; A61B 8/403

(57) ABSTRACT

Diagnostic imaging system and method comprising a device for facilitating breast examinations, comprises: at least one hemispherical portion having a convex outer surface and a concave inner surface defining a containment chamber of a breast; means to wear said hemispherical portion positionable at least around the user's torso to hold the hemispherical portion on said breast; and a connecting tube associated with said hemispherical portion to put in fluid communication the containment chamber with a depressurisation source; the hemispherical portion being made of elastic material to be switchable between a compression condition of the breast and a release condition of the breast.

The present invention relates to a diagnostic imaging system comprising a device for facilitating breast examinations.

More particularly, it relates to a device for compressing the patient's breast during examinations for the early diagnosis of breast tumours carried out with the optical technology. The diagnostic system can also allow the coupling of the examination with other diagnostic techniques, such as ultrasound.

The breast tumour is the second tumour form in women after skin tumour, but the first for mortality.

A particularly disturbing phenomenon is the continuous lowering of the average age at which the tumour arises, together with the current diagnostic inadequacy of the present screening instruments in case of dense breasts, characteristic of women in pre-menopausal age and, in general, in good fitness status. In fact, mammography is not reliable enough in case of dense breasts. The ultrasound alone is likely to be inefficient if it is not “pre-directed” on the attention areas to be examined.

The current systems which are able to provide functional information or to diagnose dense breasts, such as MRI, CT, PET, are highly expensive, thus, not suitable for screening or mass diagnosis, and invasive, thus, not repeatable in short periods.

In the general context, today's diagnostic paths always involve the use of ultrasound downstream of each first not clearly negative diagnosis. This is because, today, no primary diagnosis method has both sufficient sensitivity in order to diagnose the possible malignancy or benignity of the lesion and sufficient specificity in order to precisely locate it.

The optical technique used nowadays, consists in the analysis of changes in the concentration of deoxyhemoglobin in the breast tissue.

In particular, it is known to illuminate the breast with a red light at 640 nm and detect the light attenuation values with a video camera, opposed to the light source, to analyse variations in the concentration of deoxyhemoglobin in the capillaries. This allows the detection of the existence of “neoangiogenesis” areas, i.e. areas of abnormal vascularization generated to feed the tumour cells, regardless of the density of the tissue to be analysed. This technique overcomes the important current limitations: its prerogative, in fact, consists in the ability to detect the existence of “neoangiogenesis” areas, i.e. areas of abnormal vascularization generated to feed the tumour cells, regardless of the density of the tissue to be analysed.

As an example of application of such technique, the document U.S. Pat. No. 6,587,578 describes an optical mammoscopy system (DFOM) in which a breast supporting device is used, which is arranged between a fixed support and a flexible membrane which is part of a compression system. In particular, the membrane rests on the breast and an external inflation system pushes the membrane in direct contact with the breast providing it a slight pressure.

However, such mammoscopy system presents important drawbacks mainly related to the overall dimensions of the device.

Furthermore, such device turns out to be structurally complicated and, thus, difficult to manage.

Another important drawback of the known art comes from the inconvenience of use by the patient who must access a fixed area in which it engages the breast to the supporting device. Such supporting device is housed in an area of the machine, which is not adaptable to the morphology of the patient who, thus, must adapt herself, with consequent drawbacks in terms of inconvenience, in order to properly place the breast on the device.

Furthermore, the device turns out to be particularly inconvenient for the operator who cannot access areas of the breast which remains secured to the device, for example to carry out further controls with ultrasonic probes.

Object of the present invention is thus to provide a diagnostic imaging system comprising a device for facilitating breast examinations, which is able to solve the drawbacks of the known art, described above.

In particular, it is an object of the present invention to provide a device for breast examinations, which is structurally simple, easy to use, and wearable as a normal bra.

Moreover, it is an object of the present invention to provide a device, which is adaptable to the breast and in general to the morphology of the patient and which does not impede the operator access to any breast area.

Further object is to provide a device, which is able to provide a slight pressure in a uniform manner over the entire breast surface.

Other object is to provide a disposable device.

Other object is to provide a system and its diagnostic method, which has an easy and immediate application.

In accordance with the present invention, such and other objects are achieved by a device for facilitating breast examinations and by a system and its diagnostic imaging method comprising the technical characteristics exposed in one or more of the appended claims.

There are several advantages related to this solution compared to the solutions of the known art.

The positioning is achieved by the application of a negative pressure, i.e. by sucking the air around the breast and by collapsing a simple membrane on it, as a second layer of skin, preferably a silicone “disposable” membrane, which ensures the maximum hygiene.

This setting ensures a comfortable complete immobilization of the organ and allows the execution of a reliable examination.

Furthermore, the slight pressure exerted by the atmospheric pressure on the membrane and, consequently, on the breast results in a momentary block of the microcirculation in the capillaries, whose different recovery times (a few seconds) depend on the greater elasticity of normal capillaries than those neoangiogenetic, thus providing further information on the presence of a possible vessel network dedicated to feed a nascent tumour.

It is thus possible to detect the tumour looking for the vessel network that feeds it, the neoangiogenesis, which is much larger and identifiable than the tumour itself.

With this type of device it is also possible to integrated into a single apparatus test methods which are now carried out only separately in order to facilitate the making of the diagnosis; significantly reduce examination times; drastically simplify logistics thanks to the use of a single non-ionizing device, located in a room without the need for isolation from radiations (instead of two devices, one of which is based on ionizing radiation).

The characteristics and advantages of the present invention will become apparent from the following detailed description of a practical embodiment thereof, illustrated by way of non-limiting example in the accompanying drawings, in which:

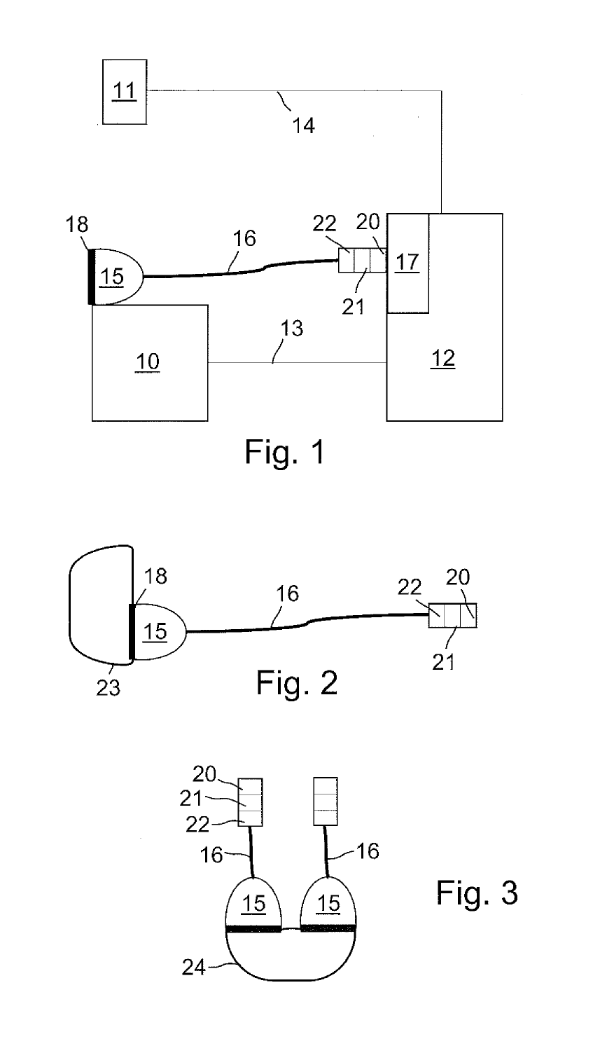

FIG. 1 shows a schematic diagnostic imaging system provided with a device for facilitating breast examination, in accordance with the present invention;

FIG. 2 shows a schematic form of a device for facilitating breast examination, in accordance with the present invention;

FIG. 3 shows a further schematic form of a device for facilitating breast examination, in accordance with the present invention.

Referring to the attached figures, a diagnostic imaging system, in accordance with the present invention, includes an illumination system 10 that emits light in the visible range, for example at 640 nm, or a light in the near infrared between 750 and 1400 nm.

A video camera 11 is opposed to the illumination system 10. The video camera 11 is arranged (distance, filters, etc.) in a known manner in order to capturing the images of the biological tissue under examination illuminated by the illumination system 10 below.

A control centre 12 is electrically connected with a cable 13 to the illumination system 10 and with a cable 14 to the video camera 11, for the control, management and transfer of data.

The breast to be analysed is inserted in a device 15 connected pneumatically with a tube 16 to a depressurisation source 17.

The device 15 is substantially a membrane made of an elastic material, for example of the silicone type.

More particularly, the material is also flexible and resilient. Moreover, it should be transparent to the light used by the illumination system 10.

In a first embodiment it is composed of a container having a substantially hemispherical shape (cup), for only one breast made of a silicone membrane having a thickness, for example, between 0.5 and 5 tenths of a millimetre. It comprises a circular edge 18, which will have to be in contact with the user's torso, of increased thickness (a few millimetres) so that it has greater strength in order to wear the device 15, without tearing it, and make sure that it is an air-tight device.

The device 15, although flexible and adaptable to different breast sizes, will have variable sizes and forms so that it can be used by users with different breast sizes.

In greater detail, the device 1 comprises: at least one hemispherical portion having a convex outer surface and a concave inner surface defining a chamber for containing a breast.

Means 23 to wear the hemispherical portion are also provided, which can be positioned at least around the user's torso to maintain he hemispherical portion on said breast. In this situation, the connection tube 16 is associated with the hemispherical portion to put in fluid communication the containment chamber with the depressurization source. In this way, the hemispherical portion, which, as described above, is made of elastic material, is switchable between a breast compression condition in which the inner surface adheres to the breast and the containment chamber has a corresponding negative pressure relative to the pressure of the outer surface, and a release condition of the breast in which the inner surface does not adhere to the breast and the containment chamber has a pressure equal to the pressure at the outer surface.

Preferably, the hemispherical portion is made of a material transparent to electromagnetic radiation included in the visible spectrum and/or to electromagnetic radiation included in the infrared spectrum.

Advantageously, the hemispherical portion is made of a material transparent to ultrasonic frequencies.

In this way, the hemispherical portion allows both the passage of visible and infrared light, for the optical analysis carried out by the video camera 11, and the ultrasound passage for the analysis carried out by an ultrasound scanner.

For this purpose, the hemispherical portion comprises landmarks.

With reference to FIG. 3, there are two hemispherical portions, placed side by side and each of which is associable to a respective breast.

The means 23 to wear the hemispherical portion (not illustrated and described in detail since of known type), include at least one band having respective ends engaged to opposite areas of the hemispherical portion.

The band surrounds the trunk and/or the user's shoulder for maintaining the hemispherical portion fitted on the breast.

More particularly, the means 23 to wear the hemispherical portion preferably comprise a plurality of bands each of which is made of elastic material and adaptable to the user's sizes. In this way, once the hemispherical portion has been worn, the user is free to move without disturbing the correct positioning of the device 15 with respect to the breast.

As specified above, the tube 16 connects the device 15 to the depressurisation source 17. The tube 16 is connected to the device 15 in any one of its point but preferably in a point, which minimize the hindrance, is placed in the most external point of the device 15 with respect to the user's body.

The tube 16 is connected to the depressurisation source 17 via a connector 20.

The depression created by the depressurisation source 17 is very low and it is comprised between 5 and 10 mmHg and preferably is comprised between 0 and 80 mmHg.

The tube 16 also includes a filter 21 to prevent the entering of dust and liquid coming from the device 15 into the depressurisation source 17.

The tube 16 possibly comprises also a non-return valve 22, so that even in case the tube 16 comes off from the depressurisation source 17, the device 15 is however maintained in depressurisation.

The device 15, the tube 16 including the connector 20, the filter 21 and the valve 22 are preferably of the disposable type, for hygienic reasons.

It is also possible to use two devices 15 joined together with bands 24 to wear them as normal bras.

The control centre 12, which includes, among other things, a computer, completely manages the diagnostic imaging system in a known manner. Other diagnostic imaging techniques may be used in place of or in combination with the previous one.

The operation of the invention is apparent for the skilled in the art from what has been described and, in particular, it is the following.

The hemispherical portion of the device 15 has to be placed on the user's breast, making sure that the edge 18 adheres properly on the torso of the user. In this situation, the containment chamber is fitted on the breast.

The size of the device 15 should be slightly greater than the size of the breast to be analysed so that it can be fitted properly.

Therefore, the user wears the device 15 by means of the aforementioned elastic bands.

Subsequently, the illumination system 10 is activated and the breast is positioned in correspondence with a diagnostic area comprised between the illumination system 10 and the video camera 11.

Such diagnostic area provides a supporting surface under which the illumination system 10 develops and which thus provides the illumination of the breast.

The position of the breast is thus controlled through the image acquired by the video camera in order to carry out a first calibration procedure of the illumination system 10 with respect to the breast.

At this point, at least a pressure difference is applied between the inner chamber and the outer surface of the hemispherical portion, generating a negative pressure in the chamber.

This pressure difference switches the hemispherical portion in a compression condition of the breast in which the inner surface of the portion adheres to the breast.

In this way, the depressurisation source 17, upon demand of the control center 12, creates a slight depression inside the device 15, by making the membrane adhere to the user's breast and obtaining a slight surface pressure on it.

The control centre 12 activates the video camera 11 and captures the images. Such images are then processed, displayed on a first screen, stored, printed etc.

Advantageously, as a result of the step of the image displaying on the first screen, it is possible to carry out a further acquisition step of a series of images by means of an ultrasound scanner and display such images on a second screen.

In particular, a suitable number of landmarks are applied to the device 15 or the illumination system 10, e.g. realized by using optical, magnetic, radio frequency etc. technology.

Such additional step of image acquisition by means of an ultrasound scanner is carried out in case suspicious areas are detected by acquiring images with the video camera. In fact, an ultrasound is immediately carried out in these suspicious areas thanks to the possibility to directly intervene on the breast with the ultrasound probe in any point of the breast. Such advantage is determined by the absence of obstacles and thus by the possibility for the operator to move on the sides or frontally with respect to the breast.

As regards the step of activating the illumination system (10), the breast is advantageously illuminated at an electromagnetic frequency included in the visible spectrum by emitting an electromagnetic radiation at a wave length preferably equal to 640 nm. Alternatively, the illumination system (10), can emit an electromagnetic radiation included in the infrared spectrum, preferably at a wave length comprised between 750 and 1400 nm.

Preferably, the method of the present invention is carried out by a plurality of steps of applying a pressure difference in which each step is continued for a respective predetermined period of time. For this purpose the pressure turns out to be variable in time to define alternated sequences of steps in which a determined depression is realized for a predetermined period.

In particular, a first step of applying a pressure difference inside the chamber at a value lower than 8 mmHg, preferably of 5 mmHg is carried out.

Subsequently, a second step of applying a pressure difference at a value greater than 9 mmHg, preferably of 10 mmHg is carried out.

Finally, the pressure difference inside the chamber is brought back to the value lower than 8 mmHg.

The step of image acquisition is carried out by the sub-steps of: acquiring a first series of reference images between the first and the second step of applying a pressure difference by means of the video camera; subsequently, acquiring a second series of images, obtained activating the illumination system from different points with respect to the breast, between the second and the third step of applying a pressure difference.

In this way, the second series of images is processed by the control centre (12) to detect suspicious areas.

Moreover, means to fix the device 15 to the illumination system 10 are possibly applied in order to prevent movements of the device 15 itself.

The fixing means are realized e.g. through buttons, clip, velcro, specific connectors, etc.

The so-conceived system is susceptible of numerous modifications and variations, all falling within the scope of the inventive concept; moreover all details can be replaced by technically equivalent elements.

In fact, it was described a diagnostic imaging system comprising a device for facilitating breast examinations, but such technique may be applied also to other biological objects.

1. Device for facilitating breast examinations, characterized in that it comprises:
at least one hemispherical portion having a convex outer surface and a concave inner surface defining a containment chamber of a breast;
means to wear said hemispherical portion, which can be positioned at least around the user's torso to maintain the hemispherical portion on said breast; and
a connection tube associated with said hemispherical portion to put in fluid communication the containment chamber with a depressurization source;
said hemispherical portion being made of elastic material to be switchable between a breast compression condition in which the inner surface adheres to the breast and the containment chamber has a corresponding negative pressure relative to the pressure of the outer surface, and a release condition of the breast in which the inner surface does not adhere to the breast and the containment chamber has a pressure equal to the pressure at the outer surface.

2. Device according to claim 1, characterized in that said hemispherical portion is made of a material transparent to electromagnetic radiation included in the visible spectrum.

3. Device according to claim 1, characterized in that said hemispherical portion is made of a material transparent to electromagnetic radiation included in the infrared spectrum.

4. Device according to claim 1, characterized in that said hemispherical portion is made of a material transparent to the ultrasonic frequencies.

5. Device according to claim 1, characterized in that said hemispherical portion comprises landmarks.

6. Ultrasonic device according to claim 1, characterized in that it comprises a pair of hemispherical portions, placed side by side and each of which is associable to a respective breast.

7. Device according to claim 1, characterized in that said means to wear the hemispherical portion comprise at least one band having respective ends engaged to opposite areas of said hemispherical portion; said band surrounding the trunk and/or the user's shoulder.

8. Device according to claim 7, characterized in that said means to wear the hemispherical portion comprise a plurality of bands each of which made of elastic material and adaptable to the user's sizes.

9. Diagnostic imaging system comprising:
an illumination system (10);
a video camera (11);
a control centre (12) for managing said illumination system (10) and said video camera (11);
characterized in that it comprises at least one device (15) in accordance with claim 1, into which the breast to be examined is inserted; and
a depressurisation source (17) connected to said connection tube of the device to switch the hemispherical portion between the respective compression condition and the release one.

10. System according to claim 9, characterized in that said video camera (11) is positioned opposite the illumination system (10).

11. System according to claim 9, characterized in that said depressurisation source (17), in the compression condition of the hemispherical portion, generates a negative pressure in the containment chamber of the breast comprised between 0 and 80 mmHg, and more preferably between 5 and 10 mmHg.

12. System according to claim 9, characterized in that it further comprises an ultrasound scanner having a probe identified with landmarks.

13. System according to claim 12, characterized in that it comprises at least a first displaying screen of the images acquired by the video camera (11) and at least a second displaying screen of the images acquired by the ultrasound scanner; said screens being spaced between each other to be visible from respective opposite sides.

14. Diagnostic imaging method for facilitating breast examinations comprising the steps of:
wearing a device by fitting a containment chamber defined by a hemispherical portion of said device on a respective breast;
activating an illumination system (10);
positioning the breast in correspondence with a diagnostic area comprised between the illumination system and a video camera;
applying at least one pressure difference between the inner chamber and the outer surface of the hemispherical portion, generating a negative pressure in said chamber;
said pressure difference switching the hemispherical portion in a compression condition of the breast in which the inner surface of the portion adheres to the breast;
activating a video camera (11);
acquiring a series of images from said video camera (11) by means of a control centre (12); and
displaying such images on a first screen.

15. Method according to claim 14, characterized in that, subsequently to the step of displaying the images on the screen, the step of acquiring a series of images is carried out by means of an ultrasound scanner together with the displaying of such images on a second screen.

16. Method according to claim 14, characterized in that said step of wearing the device is carried out by tying the elastic bands around the body to position the hemispherical portion around the breast.

17. Method according to claim 14, characterized in that said step of activating an illumination system (10) is carried out by illuminating the breast at an electromagnetic frequency included in the visible spectrum and/or included in the infrared spectrum.

18. Method according to claim 17, characterized in that said step of activating the illumination system (10) is carried out by emitting an electromagnetic radiation at a wave length equal to 640 nm and/or emitting an electromagnetic radiation at a wave length comprised between 750 and 1400 nm.

19. Method according to claim 14, characterized in that it comprises a plurality of steps of applying a pressure difference; each step being carried out for a respective predetermined period of time.

20. Method according to claim 19, characterized in that it comprises:
a first step of applying a pressure difference inside the chamber at a value lower than 8 mmHg; subsequently
a second step of applying a pressure difference at a value greater than 9 mmHg; and subsequently
a third step of applying a pressure difference inside the chamber at a value lower than 8 mmHg;
said steps being spaced between each other for a determined period of time.

21. The method according to claim 20, characterised in that said step of acquiring a series of images comprises the sub-steps of:
acquiring a first series of reference images between the first and the second step of applying a pressure difference; subsequently
acquiring a second series of reference images between the second and the third step of applying a pressure difference by activating the illumination system from different point with respect to the breast;
said second series of images being processed by the control centre (12) to detect suspicious areas.

22. Method according to claim 21, characterized in that said step of acquiring a series of images further comprises the sub-step of acquiring a third series of images using an ultrasound scanner following the third step of applying the pressure difference.
